(19) United States
(12) Patent Application Publication
(10) Pub. No.: US 2015/0104425 A1
(43) Pub. Date: Apr. 16, 2015

(54) Medical food for high cortisol breast cancer

(71) Applicants: John Edgar Menear, Santa Cruz, CA (US); Joseph Ramaekers, Aptos, CA (US)

(72) Inventors: John Edgar Menear, Santa Cruz, CA (US); Joseph Ramaekers, Aptos, CA (US)

(73) Assignee: CortControl, Inc., Santa Cruz, CA (US)

(21) Appl. No.: 14/544,279

(22) Filed: Dec. 17, 2014

Related U.S. Application Data
(63) Continuation-in-part of application No. 13/843,581, filed on Mar. 15, 2013.
Application No. 13/843,581 is a continuation-in-part of application No. 13/718,319, filed on Dec. 18, 2012.
Application No. 13/718,319 is a continuation-in-part of application No. 12/631,745, filed on Dec. 4, 2009, now abandoned.
Application No. 12/631,745 is a continuation of application No. 11/492,464, filed on Jul. 24, 2006, now abandoned.
(60) Provisional application No. 61/964,100, filed on Dec. 24, 2013.

(51) Int. Cl. A61K 38/17; A23L 1/305; A23L 1/30; A61K 35/74; A61K 31/716

(52) U.S. Cl. CPC A61K 38/1709; A61K 35/74; A61K 31/716; A23L 1/3014; A23L 1/305; A23V 2002/00
USPC 424/93.4

(57) ABSTRACT

A method of increasing effectiveness of breast cancer treatment or recovery using a medical food. This breast cancer medical food consists of transfer factor, lactic acid generating bacteria, and/or glucans in appropriate combinations. The breast cancer medical food, administered correctly, reduces cortisol levels and enhances immune function. This method is applied to breast cancer patients with high cortisol levels. Dosage amounts are adjusted for client weight. Consumption frequency and dosage may be adjusted in response to cortisol measurements and killer T-cell-counts. Typically, consumption of the breast cancer medical food is done under professional supervision, and may be combined with other treatment strategies.

### CROSS-REFERENCE TO RELATED APPLICATIONS

The current application is a continuation-in-part of non-provisional application U.S. Ser. No. 13/843,581, filed Mar. 15, 2013, which is a continuation-in-part of application Ser. No. 13/718,319, filed on Dec. 18, 2012. Application Ser. No. 13/718,319 is a continuation-in-part of application Ser. No. 12/631,745, filed Dec. 4, 2009 and a continuation-in-part of application Ser. No. 11/492,464, filed Jul. 24, 2006. The current application also claims priority to U.S. provisional application 61/964,100 filed Dec. 24, 2013. The above references are incorporated by reference in their entirety into the current application.

### STATEMENT REGARDING FEDERALLY SPONSORED RESEARCH

Not Applicable

### REFERENCE TO A MICROFICHE APPENDIX

Not Applicable

### BACKGROUND OF THE INVENTION

1. Field of the Invention

This invention relates to the treatment of human breast cancer, which is correlated with an excess of cortisol (corticosteroids). More specifically, this invention describes method of feeding a medical food composition, which mitigates breast cancer by reducing a patient's cortisol level. An estimated 232,340 new cases of invasive breast cancer and 39,620 breast cancer deaths were expected to occur among U.S. women in 2013. About eight in ten breast cases and nearly nine in ten breast cancer deaths will occur among women aged 50 years and older.

2. Description of Related Art

The connection between high cortisol levels, breast cancer incidence, and breast cancer mortality has been established.

Stanford researchers (David Spiegel's team) report that women with advanced breast cancer who have abnormally high daytime levels of cortisol are significantly more likely to die sooner than patients with normal levels of the hormone.

Disruption of cortisol's diurnal rhythm is also a correlated factor. In the study, swing shift workers experienced an increased incidence of breast cancer.

The study involved 104 San Francisco Bay Area women with metastatic cancer, or disease that had spread beyond the breast. The researchers extracted cortisol from the saliva of the volunteers, which was taken at regular intervals during the course of three days. They then observed the women's daytime rhythms as reflected in their cortisol levels, which are generally high in the morning and low in the evening. The researchers found that some of the study volunteers had abnormal patterns of cortisol secretion. Sometimes, cortisol levels remained relatively flat, rather than high in the morning and low in the evening. Other volunteers exhibited cortisol peaks at unexpected times.

The researchers also found that women with abnormal cortisol levels had fewer immune system cells known as killer T-cell count, and this reduced immunity was associated with higher mortality.

Supporting work from the University of California at Davis stated, “There are now sufficient data to support the notion that the loss of normal diurnal variation in cortisol is another possible mechanism that could account for the observed association between shift work and breast cancer incidence.”

Typical breast cancer treatments utilize chemotherapy, radiation, and surgery. During treatment, the number of natural killer cells typically drops. Both high cortisol levels as well as the treatment itself contribute to the reduced killer T-cells. Sometimes, treatment has to be delayed because the killer T-cells are too low.

Following chemotherapy, radiation and surgery, the patient usually feels exhausted and depleted. The recovery process is usually difficult.

Mammal studies have demonstrated that consuming a combination of transfer factor and lactic acid generating bacteria confers two benefits. First, the immune system is enhanced. Second, cortisol levels are lowered. Immune system enhancement was confirmed by measuring the increase of killer T-cells. Cortisol lowering was measured in studies of cattle, where mortality decreased and fertility increased.

Adding glucans to the combination of transfer factor and lactic acid generating bacteria increases the benefits. Glucans may be present as natural or hybrid mushrooms.

Breast cancer treatments (chemotherapy, radiation, and surgery) are intrusive at best. A food based approach (alone or in combination with other therapies) would be desirable. The side effects of medical foods are far less than conventional therapies.

A food based treatment option is appropriate and desirable. Normally, a food based treatment augments—rather than replaces—conventional therapy.

The consuming public now understands that foods possess more than basic nutrition (protein, carbohydrate, fat, etc.). For example, 95% of consumers agree that “certain foods have health benefits that go beyond basic nutrition and may reduce the risk of disease or other health concerns”. More than 50% of consumers believe that foods can replace the use of drugs.

The Federal Drug Administration acknowledges this trend with the relatively new category of “Medical Foods”. Medical Foods should be administered or supervised by a doctor, nutritionist, nurse, medical technician or equivalent health care professional. Medical foods are defined by the Federal Drug Administration in 21 CFR 101.9(j)(8).

A medical food that reduces cortisol and builds immunity is needed as a stand-alone treatment or as an enhancement to conventional breast cancer strategies.

### BRIEF SUMMARY OF THE INVENTION

Following is a condensed summary of the invention. By necessity, details are omitted in order to simply state the essence of the invention. Omitted details within this section should not be construed in a way that limits the scope of the invention.

The instant invention is a method of treating breast cancer with a medical food that reduces human cortisol levels and simultaneously builds the immune system. The method applies to patients during conventional medical treatment and during the recovery period.

This method is directed to breast cancer patients where high cortisol is present or to patients where the cortisol diurnal cycle is irregular. If high cortisol is present, the disclosed treatment method is within the scope of this instant invention.

The instant invention does not claim to address all breast cancer. The invented method addresses breast cancer where high cortisol is a positively correlated issue. It also addresses breast cancer where high cortisol appears in concentrated spikes throughout the day.

This method involves feeding a breast cancer patient a combination of (1) transfer factor and lactic acid generating bacteria or (2) transfer factor, lactic acid generating bacteria, and glucans. Throughout this disclosure, the term “breast cancer medical food” will refer to a combination of (1) transfer factor and lactic acid generating bacteria or (2) transfer factor, lactic acid generating bacteria, and glucans.

Cortisol reduction with the breast cancer medical food has been demonstrated in mammals by cortisol sampling. Refer to FIG. 1 and FIG. 2.

Anecdotal studies also indicate that consuming breast cancer medical food helps during recovery. Recovering patients experience more energy, better sleep, less depression and fewer colds. These metrics are self-reported, but the preponderance of agreement is probative.

In this disclosure, high cortisol is defined as “above the United States population average”, when measured as salivary, serum, hair, or urine cortisol.

The breast cancer medical food contains at least transfer factor and lactic acid generating bacteria. Any breast cancer food treatment that includes consumption of transfer factor and lactic acid generating bacteria is within the scope of this invention. A breast cancer treatment that does not include consumption of transfer factor and lactic acid generating bacteria is outside the scope of this invention. A breast cancer medical food may include other components in conjunction with transfer factor and lactic acid generating bacteria.

The inventors have found through field trials that feeding a mixture of transfer factor and lactic acid generating bacteria lowers cortisol in mammals. Addition of glucans further enhances the cortisol-lowering effect. The cortisol-reducing property was first determined during controlled cattle studies. Diseased cattle showed significant health improvement and reduced cortisol, relative to a control group.

During the cattle studies, blood cortisol was measured over time as a chemical marker. As the animals improved, decreased cortisol levels were observed. Cortisol levels in the test cattle (calves) decreased from 34.3 to 21.5 ng/ml. Cortisol levels in the control calves did not decrease.

Although supporting data involves cattle, application to humans is supported. Cross-over charts show that the biochemical response of cattle and humans is similar. Further, transfer factor confers benefits across species. For instance, bovine transfer factor builds immunity on dogs and humans, and ovum transfer factor builds immunity on cattle and humans.

A cortisol-lowering response in humans is observable via dietary inclusion of transfer factor and lactic acid generating bacteria.

For the current method-of-use invention, the central underlying facts are (1) ingesting a breast cancer medical food containing transfer factor and lactic acid generating bacteria leads to reduced human cortisol plus improved immune function, (2) high cortisol correlates to breast cancer mortality, and (3) improved immune function benefits the patient during treatment and during recovery.

Lowering cortisol also improves sleep because high cortisol is a cause of insomnia. This is an extra benefit because restful sleep is generally helpful to a healing process.

This application is not limited by any specific mechanism that explains how cortisol is lowered or how immune function is increased. Claims are based on observation rather than theory. Claims are based on a breast cancer medical food formulation that lowers cortisol, increases immune function, and supports a breast cancer patient—regardless of the underlying mechanism.

Glucans may be added to the combination of transfer factor and lactic acid generating bacteria to increase effectiveness.

Other useful additives include GABA. Gamma amino butyric acid (GABA) is the brain's primary inhibitory (i.e., calming) neurotransmitter. It neutralizes adrenaline as a primary function. Serotonin and melatonin are also appropriate additives in some cases.

There may be many reasons and many factors related to breast cancer. The instant application method provides a useful remedy where high cortisol is a positively correlated factor.

A breast cancer medical food that mitigates the symptoms of breast cancer by lowering cortisol levels and improving immune function is described herein. Administration by feeding is discussed in this disclosure. However, it would be obvious to a person of ordinary skill that other forms of administration—such as injection, capsule swallowing or intravenous administration—would lead to the same result.

Objects of this invention include some or all of the following: (1) improve mortality and recovery with a breast cancer medical food, (2) combine transfer factor and lactic acid generating bacteria to create a breast cancer medical food, (3) add glucans to the combination of transfer factor and lactic acid generating bacteria, (4) select the proportion of each breast cancer medical food component within each dosage, (5) select the dosage level based on feeding frequency and patient weight, (6) allow dosage adjustments over time based on cortisol measurements, (7) allow dosage adjustments over time based on killer T-cell measurements, and (8) allow dosage adjustments over time based on killer T-cell measurements.

### BRIEF DESCRIPTION OF THE SEVERAL VIEWS OF THE DRAWINGS

FIG. 1 shows evening effect of the invented breast cancer medical food from a cattle study. Note that cortisol levels decreased.

FIG. 2 shows morning effect of the invented medical food from a cattle study. Note that cortisol levels decreased.

### DETAILED DESCRIPTION OF THE INVENTION

A composition of transfer factor and lactic acid generating bacteria was patented by Joseph Ramaekers (a current joint inventor). Refer to U.S. Pat. No. 6,962,718, claim 6, issued Nov. 8, 2005, which recites, “A formulation comprising pharmaceutically acceptable transfer factor and a pharmaceutically acceptable lactic acid generating bacteria wherein the amount of said transfer factor is from 10 mg to 10,000 mg per ounce of formulation”.

This composition has been used successfully as a medical food in the veterinary field for a variety of animal diseases. For example, feedlot cattle showed decreased mortality. Mammal fertility was increased. Photographs clearly document decreased tumor size in dogs and horses.

One benefit of this breast cancer medical food formulation is cortisol reduction. Cortisol is the predominant stress hormone. A second benefit is immune system building.

High levels of cortisol correlate to higher mortality rates of breast cancer patients. This is particularly important during the recovery period. There is also evidence that high cortisol baseline levels and irregular cortisol rhythms contribute to breast cancer incidence. Reduced incidence (as relapse) is also pertinent to the recovery period.

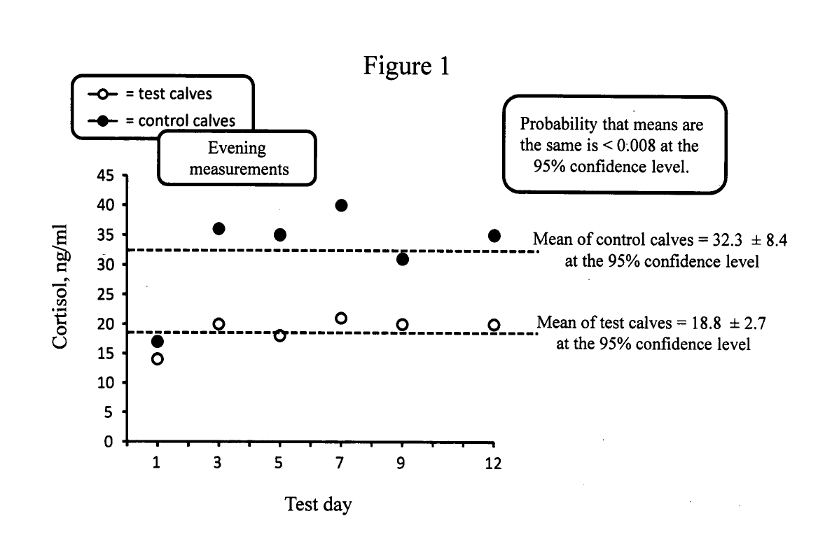

FIG. 1 summarizes the evening cortisol reduction (test versus control) for a calf study over a 12 day period. Our more current research indicates that humans experience a similar evening cortisol reduction after consuming the breast cancer medical food.

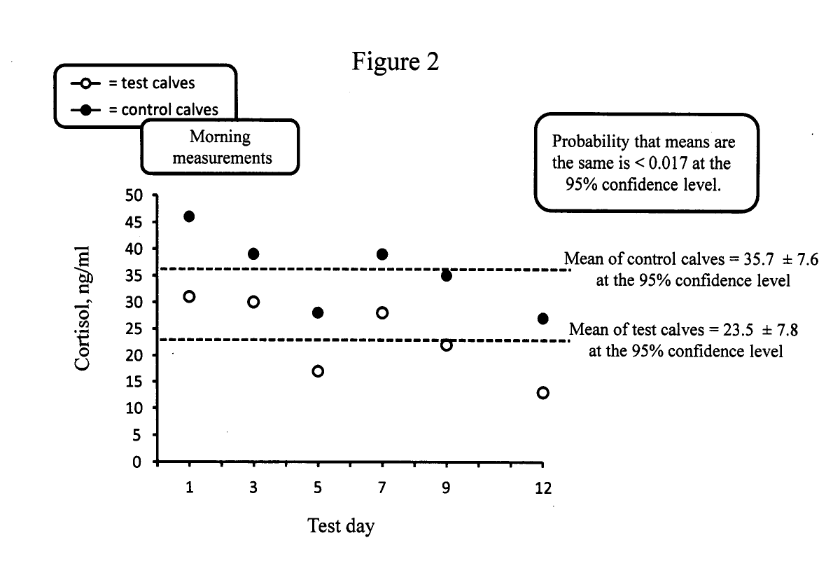

FIG. 2 summarizes the morning cortisol reduction (test versus control) for a calf study over a 12 day period. Our more current research indicates that humans experience a similar morning cortisol reduction after consuming the breast cancer medical food.

Laboratory tests have demonstrated the immune building properties of combining transfer factor, lactic acid generating bacteria, and glucans. A breast cancer medical food combination is capable of increasing the immune system more than five times the baseline level. The metric to demonstrate this five-fold increase was killer T-cell count.

The instant method-of-use invention is designed for people diagnosed with breast cancer, where high cortisol is a positively correlated factor. An ingested breast cancer medical food reduces human cortisol and increases immunity.

A breast cancer medical food is designed to help people during and after conventional breast cancer treatments. Conventional breast cancer treatments include radiation, surgery, chemotherapy or combinations thereof.

Breast cancer has multiple causes. Not all involve high cortisol. But if a person has both high cortisol and breast cancer, that person can be treated via this current invention. Medical foods are usually used to supplement conventional therapies.

Components of the breast cancer medical food include transfer factor and lactic acid generating bacteria. Glucans may be added. Following is a description of each component.

Transfer factor is produced by leucocytes and lymphocytes. Transfer factor comprises small water soluble polypeptides of about 44 amino acids that stimulate or transfer cell mediated immunity from one mammal to another.

The properties, characteristics and processes for obtaining transfer factor are discussed in U.S. Pat. Nos. 4,816,563; 5,080,895; 5,840,700, 5,883,224 and 6,468,534, the contents of which are hereby incorporated by reference into the present application.

Alternative sources of transfer factor include avian transfer factor, ova transfer factor, and colostrum from goats, pigs, horses and humans. This listing is not complete. In addition, combinations of transfer factors from any number of sources may be used in human formulations.

In breast cancer medical foods, a significant fraction of transfer factor has a molecular weight of less than 10,000 Daltons. Transfer factor may be derived from colostrum, but it is considerably different from colostrum. It takes approximately 1000 grams of colostrum to produce 1 gram of the transfer factor used for a breast cancer medical food.

Transfer factor is commercially available, and known to be safe. Transfer factor is included in the Physician's Desk Reference.

Lactic acid generating bacteria is a necessary component of a breast cancer medical food, and is GRAS (generally recognized as safe). Lactic acid generating bacteria support digestion and brain health. Lactic acid generating bacteria provide healthful effects that are found yin non-pasteurized sauer kraut and cod liver oil. Within the intestinal tract, lactic acid generating bacteria are beneficial. It has been estimated that 80% of human health depends on beneficial intestinal bacteria. Intestinal bacteria account for 90% of the total human body cell count.

Lactic acid generating bacteria is only one component of the breast cancer medical food, but it is a necessary inclusion.

A human body becomes stressed by poor digestion, and cortisol levels will increase to reflect that stress. Stated differently, lactic acid generating bacteria contributes to cortisol reduction via improved digestion.

Glucans (polysaccharides) are known to support the immune system. But when combined with transfer factor and lactic acid generating bacteria, a synergy is created. The combined effect on breast cancer is greater than the effect predicted from summing individual components. Glucans may be present as natural or hybrid mushrooms.

The method of using transfer factor, lactic acid generating bacteria, and glucans for treating breast cancer can also be viewed as two improvements to U.S. Pat. No. 6,962,718. The two improvements (limitations) are (1) the addition of glucans, to the patented formulation, and (2) the specific method of using the patented formulation to support a breast cancer patient during treatment and recovery, regardless of treatment type. Improvement claims are presented in the claims section.

A breast cancer medical food composition may be augmented with additional additives. Example additives are minerals, probiotics, prebiotics, dimethyl glycine, ascorbic acid, Vitamin A, Vitamin B1, Vitamin B2, Vitamin B12, Vitamin D3, Vitamin E, dipotassium phosphate, potassium chloride, magnesium sulfate, calcium pantothenate, minerals, antioxidants, amino acids, nutraceuticals, inositol hexaphosphate (Ip6), mannans, olive leaf extract, and phytosterols. In certain preferred embodiments, mannans are derived from Aloe Vera. In certain preferred embodiments, phytosterols may be derived from soya bean.

Probiotic additives include, but are not limited to B. subtlis, B. longum, B. thermophilium, B. coagulans, E. faecium, and S. cerevisia, L. casei, L. plantarum, Pediococcus acidilacticii, Kluyveromyces marxianus fragillis and combinations thereof

The above listing does not include all possible additives. A breast cancer medical food may also include one or more of the following: carrier proteins such as serum albumin; buffers such as sodium acetate; fillers such as microcrystalline cellulose, lactose, corn and other starches; binding agents; sweeteners and other flavoring agents; coloring agents; and polyethylene glycol. Additives are well known in the art, and are used in a variety of formulations.

Neurotransmitter precursors such as GABA, tryptophan, and/or melatonin may be added to a breast cancer medical food.

Relative proportions of transfer factor, lactic acid generating bacteria, and glucans within a breast cancer medical food may vary widely. Also, dosage levels can be adjusted. The reasons for the wide variation are (1) that humans can create cortisol by imagining threatening scenarios, and (2) that different stages of breast cancer treatment are involved.

With variations understood, reasonable weight ranges for transfer factor are 0.05-50 mg per pound of body weight. Reasonable weight ranges for lactic acid generating bacteria are 0.47-10 mg per pound of body weight, based on a nominal live count of 1.5×10⁶−3.5×10⁶ CFU/ounce (colony-forming-units-per-ounce). Reasonable weight ranges for glucans are 0:1-10 mg per pound of body weight.

A method of using the breast cancer medical food may have some or all of the following steps:

(1) determine that a high cortisol baseline and breast cancer are both present,

(2) determine that high cortisol spiking and breast cancer are both present,

(3) select the correct proportion of transfer factor, lactic acid generating bacteria, and glucans,

(4) choose the correct breast cancer medical food dosage level,

(5) select a feeding frequency between five times per day and once per week,

(6) begin consumption of the breast cancer medical food,

(7) periodically measure serum or salivary cortisol levels, and adjust the breast cancer medical food dosage,

(b 8) periodically measure killer T-cell count, and adjust the breast cancer medical food dosage,

(9) consume the breast cancer medical food during chemotherapy, radiation or surgery,

(10) continue consuming the breast cancer medical food after chemotherapy, radiation or surgery,

(11) reduce the dosage level after the treatment stage,

(12) extend consumption during the recovery period or beyond to prevent a relapse, and

(13) monitor progress through a health care professional.

For some patients, transfer factor, lactic acid generating bacteria, and glucans are taken together. For other patients, transfer factor, lactic acid generating bacteria, and glucans are taken at different times during the day or week. Component separation and consumption at different times are within the scope of this invention. Separate consumption and was recited in Ramaekers' U.S. Publication 20070128253, which benefits this application. The human body performs the mixing.

The physiology between animals and humans is similar. But methods of treatment differ for humans from animals. Two differences involve (1) treatment durations and (2) feeding frequency. Humans typically have a wider range of feeding frequencies, and longer treatment durations than livestock or pets. This reflects the underlying treatment guidelines.

Method steps in this application differ from animal applications, for which filing benefit is claimed. Human patients respond to a cortisol reducing food in different time frames. Treatment periods vary.

For livestock, the guideline is economic. Feeding is operationally fixed. Changing the feeding frequency affects a rancher's profit. And every livestock animal in the herd gets roughly the same dosage for the same length of time.

For humans, the guideline is effectiveness. Cortisol levels increase in response to stressful thoughts, and subside with calming thoughts. This is a significant consideration for someone with breast cancer. Hence, longer treatment periods for humans are often used, relative to animals.

Several small medical food portions per day—rather than 1 large portion per day—is not difficult for humans to arrange. Human consumption frequency may vary between five times per day and once per week.

Periodically measuring cortisol and killer T-cell count during treatment and recovery is recommended. Based on periodic measurement, dosages, frequency of feeding, and component proportions are optimized. Without measurement, the treatment program will be less exact. With measurement, treatment aspects can be better refined.

Minor variations on this disclosure remain within the scope of this disclosure.

The invention claimed is:

1. A food-based method for improving conventional treatment for a human with breast cancer plus high cortisol comprising:
combining transfer factor and lactic acid generating bacteria to create a breast cancer medical food, where
said transfer factor includes polypeptides with a molecular weight below 10,000 Daltons;
feeding said medical food to said human, where
a dosage level is chosen based on said human's weight, and
the frequency of said feeding is between five times per day and once per week; and
continuing said feeding throughout the period of said conventional treatment,
wherein said conventional treatment includes chemotherapy, radiation, or surgery.

2. The food-based method of claim 1 wherein said high cortisol is defined as a cortisol level that exceeds the human population average.

3. The food-based method of claim 1 wherein glucans are added to said medical food.

4. The food-based method of claim 3 wherein said glucans are derived from natural or hybrid mushrooms.

5. The food-based method of claim 1 further comprising:
measuring cortisol levels periodically.

6. The food-based method of claim 5 further comprising:
adjusting said dosage levels based on said measuring.

7. The food-based method of claim 3 further comprising:
changing the relative proportions of transfer factor, lactic acid generating bacteria, and glucans included within each said food dosage.

8. The food-based method of claim 1 wherein said transfer factor in each said dosage is present at 0.05 to 50 mg per pound of human body weight.

9. The food-based method of claim 1 wherein said lactic acid generating bacteria in each said dosage is present at 0.47 to 10 mg per pound of human body weight.

10. The food-based method of claim 9 wherein said lactic acid generating bacteria has a live count of 1.5×10⁶−3.5×10⁶ million colony forming units per ounce.

11. The food-based method of claim 3 wherein said glucans in each said dosage is present at 0.1 to 10 mg per pound of human body weight.

12. The food-based method of claim 3 wherein said transfer factor, said lactic acid generating bacteria, and said glucans are separated and consumed at different times within a one week period.

13. An improvement upon U.S. Pat. No. 6,962,718, claim 6 (issued Nov. 8, 2005 to Joseph Ramaekers, a current inventor) which recites—
A formulation comprising pharmaceutically acceptable transfer factor and a pharmaceutically acceptable lactic acid generating bacteria wherein the amount of said transfer factor is from 10 mg to 10,000 mg per ounce of formulation;
wherein the improvement comprises the following method-of-use limitation, feeding said formulation to a breast cancer patient to increase effectiveness of conventional treatment or conventional treatment recovery,
wherein said breast cancer patient exhibits high cortisol levels, and
wherein said conventional treatment includes chemotherapy, radiation or surgery.

14. The improvement claim in claim 13, wherein said feeding includes at least one step selected from a group including:
(a) optimizing proportions of said transfer factor and said lactic acid generating bacteria within said formulation to create a breast cancer medical food, (b) choosing a dosage level of said formulation based on said patient's weight, (c) feeding said patient a dosage between five times per day and one time per week, (d) measuring said patient's cortisol levels periodically, (e) measuring said patient's killer T-cell count periodically (f) adjusting dosage levels based on periodic cortisol measurements, and (g) adjusting dosage levels based on periodic killer T-cell count.

15. The improvement claim in claim 13, wherein glucans are added to said formulation.

16. The improvement claim in claim 15, wherein said glucans are present between at 0.1 to 10 mg per pound of patient's body weight.

17. A food-based method for improving a post-treatment recovery period for a human with breast cancer plus high cortisol comprising:
combining transfer factor and lactic acid generating bacteria to create a medical food, where
said transfer factor includes polypeptides with a molecular weight below 10,000 Daltons;
feeding said medical food to said human, where
a dosage level is chosen based on said human's weight, and
the frequency of said feeding is between five times per day and once per week; and
continuing said feeding throughout said post-treatment recovery period.

19. A food-based method for improving breast cancer treatment for a human when high cortisol levels are present comprising:
combining transfer factor and lactic acid generating bacteria to create a breast cancer medical food, wherein
said transfer factor includes polypeptides with a molecular weight below 10,000 Daltons;
feeding said breast cancer medical food to said human, where
a dosage level is chosen based on said human's weight, and
the frequency of said feeding is between five times per day and once per week;
continuing said feeding throughout treatment and recovery; and
adjusting said dosage level in response to measurements of cortisol or killer T-cell count.

20. The food-based method of claim 19 wherein glucans are added to said breast cancer medical food.
